(19) United States
(12) Patent Application Publication
(10) Pub. No.: US 2004/0096430 A1
(43) Pub. Date: May 20, 2004

(54) Method and device for producing biological tissue in a growth chamber

(76) Inventor: Jorg Bauer, Darmstadt (DE)

Correspondence Address: M Robert Kestenbaum, 11011 Bermuda Dunes NE, Albuqueruqe, NM, 87111, US

(21) Appl. No.: 10/450,478

(22) PCT Filed: Dec. 11, 2001
(86) PCT No.: PCT/EP01/14522

(30) Foreign Application Priority Data
Dec. 12, 2000 (DE) 100 61 704.2

(51) Int. Cl. C12N 5/08

(52) U.S. Cl. 424/93.7; 435/366

(57) ABSTRACT

The invention relates to a method and a device for producing tissue in a growth chamber for transplantation into or onto a human or animal body. The invention can be used particularly advantageously for the cultivation of structured functional bones. Biological cells are applied to a growth framework and both are arranged in a growth chamber. The structured and functional cultivation is achieved by allowing biologically active stimuli, for example mechanical, electrical, magnetic, chemical, olfactory, acoustic and/or optical stimuli, to act in a specified manner, in particular at different times or in different locations or in different ways. It has been found that the action of stimuli which correspond to those to which corresponding natural tissue in the body is naturally exposed is the most suitable.

The invention relates to a method and a device generally for producing biological tissue in a growth chamber and specifically for producing biological tissue for transplantation into or onto a human or animal body.

### DESCRIPTION

In recent years, there have been rapid developments in the field of production or cultivation of biological tissue and these have opened up many new possibilities in medical therapy.

Of particular note in this connection is the production of bone replacement substances which permit healing of bone defects. This still very young medical discipline involves in particular the areas of surgery and orthopedics.

Various bone replacement substances which are used as implants or transplants are already known. These are, in descending order of their value:

i) Autologous human bone, which is perfectly biocompatible but has the disadvantage of not always being readily available and of requiring a secondary operation.

ii) Human allograft bone, which has good biocompatibility but has the disadvantage of a high risk of infection and the risk of disease transmission.

iii) Bone taken from animals, which is readily available but has the disadvantage of poor biocompatibility.

iv) Ceramic bone from animals, which is readily available, has a good structure and good biocompatibility, but is unfortunately brittle and not resorbable.

v) Ceramic bone from plants, which is readily available and has good biocompatibility, but has the disadvantage of poor strength and only moderate structural approximation.

vi) Synthetic ceramic bone, which is readily available, strong and permits great variety in material terms, but has the disadvantage of poor structural approximation and limited incorporation.

vii) Synthetic pastes, which have good compatibility and are readily available, but have the disadvantage of a complete lack of structure and strength.

In practical use on humans, autologous human bone (the so-called golden standard) takes first place, ceramic bone from animals takes second place, and synthetic pastes third place. The most serious difficulties of these most frequently used replacement substances are therefore discussed in detail below.

Implantation of autologous human bone affords by far the best treatment results. This implantation has the positive effect of immediate binding to the supply system of the surrounding bone together with the spontaneous availability of the endogenous immune system using the remodeling of the tissues.

In contrast to this is the situation at the site from which the autologous human bone is taken. Here, there are often greater difficulties than at the actual defect site. Thus, in addition to the costs of the second operating site, other problems are in particular a further risk of infection, the physical burden of the healing process, and a generally inferior bone structure. The amount of removable material is also very limited. In many cases, a number of small segments even have to be removed which, however, cannot then fulfil any biomechanical functional effects at the implantation site. Moreover, autologous human bone obtained in this way, i.e. the transplant, also has to be made to go further using other bone replacement material, in order to be sufficient for the implantation site.

The transplant is typically obtained from the operating area during the actual operation. However, if the transplant takes some time to prepare during the operation, the bioactivity gradually decreases, which can lead to devitalizing of the autologous human bone. This relegates the biologically valuable transplant to a “normal” implant which, as a result of an increased degradation reaction of the remodeling system, adversely affects the course of healing and the result of treatment.

As regards the bone replacement substances based on animal bone, the ceramics presently offer a tolerable alternative. These ceramic implants are produced in such a way that the inner structures of the bone remain fully preserved and the material composition corresponds to that of human bone. Here, the advantages of inorganic material combined with good pore structure, i.e. the trabecular arrangement, can be exploited. An advantage in this case is the primary stability, which permits immediate load-bearing after implantation. By contrast, there is the serious disadvantage that these ceramic implants have no osteogenic potential at all, that is to say they are not accepted as biomass by the body. Neither the copy of the crystal form nor the biomechanical properties correspond to the human tissue form. As a result, such an implant is therefore simply a tolerated place holder which merely provides a guide route for the actual osteogenesis which, after completion of the healing process, forms a composite with the newly formed bone. The disadvantages of the limited elasticity caused by the brittleness of the ceramic cannot be compensated.

These implants are produced in specialized works. However, in view of the natural starting material, the available implant sizes and shapes are limited. The largest available ceramic implant based on animal spongiosa presently has a volume of approximately 16 cm³.

The paste implants are presently the ones mostly mentioned in scientific discussion. Here, the chemicophysical properties of the materials are of particular significance. Thus, because of the possibility of synthesis of the substances, very good adaptation of the crystals to those of human bone is possible. The human body recognizes these crystals as building blocks for bone formation and integrates them into its own remodeling of the bone. Thus, the times required for incorporation of new bone cells are shorter and almost attain those of autologous human bone. A disadvantage in this connection are the substances which are used as stabilizers or reinforcements. These substances generally cause increased cell activity for their degradation. The lack of structure occasioned by the pasty form of the replacement substance is also to be seen as a negative factor, because this first has to be reorganized in order to generate a bone framework in the form of trabeculae. Finally, such materials have poor strength, which considerably restricts their potential use.

In addition to the above-described use of finished replacement substances, cell biology methods are in principle also known for in vitro cultivation of living tissue. Thus, for example, living skin is cultivated for burn injuries. Cartilage cells are also reproduced in molds and by means of the external growing mold take on the shape of body parts and are used as such above all in reconstructive plastic surgery. Molds such as ears or noses have today reached a quality allowing them to be implanted or onplanted.

The cultivation of bone tissue is also scientifically feasible. The cultivation of bone presently takes place in what are called cell growth chambers. Special bone cells are separated from other cells and prepared for the growth chambers. The undifferentiated cells carry within them the genetic potential for generating bone-forming cells (osteoblasts) and bone-absorbing cells (osteoclasts).

In principle, such methods can be used to generate bone material. However, a bone cultivated in this way still has considerable disadvantages. For example, although the shape of the grown bone can be defined by the external shaping, for example by a hollow mold, such a bone nevertheless has no functional mechanical construction. The cultivated bone represents only a bone mass of bone substance. This bone with its sponge-like structure can admittedly be used as bone replacement substance, but it has a tendency to be rapidly resorbed by increased remodeling, because the biomechanical properties can be formed only in the course of remodeling.

On the other hand, that of fully synthetic replacements, a method for production of structured ceramic implants is known with which it is possible to synthesize the trabecular structure of a bone. In this case the individual layers of an implant are laid one on top of another and connected to one another. Subsequent heat treatment results in a completely inorganic implant based on ceramic.

In view of their serious disadvantages, however, all of the materials and methods mentioned constitute only relatively poor compromise solutions for tissue cultivation, particularly for bone replacement.

It is therefore an object of the invention to make available a method and a device for producing biological tissue which has better biological, structural and/or mechanical properties and is as far as possible accepted as endogenous tissue by the body.

A further object of the invention is to make available a method and a device for producing biological tissue which has improved properties compared to the prior art.

A further object of the invention is to make available a method and a device for producing biological tissue, in particular a bone, which tissue or bone represents a good simulation of the endogenous human or animal tissue or bone.

A further object of the invention is to make available advantageous uses of the method according to the invention, of the device according to the invention, and of the tissue produced.

The object of the invention is achieved in a surprisingly simple manner by the subject of claims 1, 21, 38, and 44, 45, 46 and 47.

In the method according to the invention for producing or cultivating biological tissue in a growth chamber, in particular for transplantation into or onto a human or animal body, biological cells are applied to a growth framework. The biological cells and the growth framework are arranged in the growth chamber, and biologically active stimuli are exerted on the growth framework and/or on the biological cells. The application takes place preferably in or outside the growth chamber.

The produced or cultivated tissue preferably comprises bone, cartilage, blood vessels, ears, noses, skin or organ sections, including complete organs.

The invention is based inter alia on the surprising finding that a large number of stimuli, in particular physical stimuli, can be used to influence, stimulate and even control artificial tissue growth, and to cultivate active tissue.

In a further step following a first growth phase, different or at least further cells are preferably applied to the framework and/or to the grown tissue, for example in order to generate, in a second growth phase, a further tissue section different than the first one cultivated. Taking the example of production or cultivation of a bone, this means that the section of the bone providing stability and shape is preferably first cultivated, and thereafter, for example, a surrounding periosteum. It is also possible to have three or more such growth phases or cultivation phases, if appropriate with renewed cell application.

The cells, preferably undifferentiated at the start of the method, are influenced, for example in terms of their growth, by the action of a stimulus or a plurality of similar, different or varied stimuli. For example, the rate of cell division and/or the differentiation of the cells during the growth process is controlled or regulated. This is preferably done globally in the growth chamber and/or locally, in particular at different times and/or different places, for example at predetermined sites on the growth framework and/or on the cells. By means of the stimulation, not only is the form of the tissue cultivated in a predetermined way, but in addition the tissue or cell conglomerate also acquires a predeterminable structure and functionality.

The form, structure and/or functionality of the tissue to be cultivated can preferably be influenced and/or predetermined by the nature, duration and/or intensity of the stimulus or stimuli.

Another surprising finding is that especially good results are obtained if the preferably physical, preferably electrical, or chemical stimulus corresponds to or is at least similar to a stimulus to which corresponding natural tissue is naturally exposed in or on the body. Thus, for example, muscles, bone and cartilage are especially well stimulated with electrical and/or mechanical stimuli or forces, parts of the auditory apparatus with acoustic stimuli, and parts of the visual apparatus with optical stimuli, for example light impulses.

In this connection, preferably, the growth framework defines essentially only at the start of the method the inner and/or outer form of the totality of the cells from which the tissue develops.

The growth framework, the cells and/or the stimulus are preferably chosen or set in such a way that, in particular at the end of the method, it is essentially the grown tissue, and no longer the growth framework, which determines the biomechanical properties.

In a preferred development of the invention, the growth framework or support framework comprises resorbable and/or nonresorbable material. The nonresorbable material gives the grown tissue additional strength, whereas the resorbable material is superseded by the cells during the method in the chamber and/or after transplantation. In doing so, the growth framework preferably disappears completely. Alternatively, the growth framework is separated from the developed tissue before, during or after the growth process.

The growth framework for its part preferably comprises biological material or cells. Alternatively, it comprises a fleece, electrically conductive material, for example metal, on which the cells are applied or introduced. In this way, electrical stimuli are distributed effectively across the whole framework.

It is particularly advantageous to use a growth framework consisting of material that promotes cell growth, for example cellulose, starch, an alcohol compound, gel, and/or a gel-like material.

During the growth process, a growth-promoting substance is preferably added, for example bone morphogenetic protein, fibrogen and/or a genetically modifed substance.

The biological tissue is preferably provided with a depot of a pharmacologically active substance which is released during the method and/or after transplantation onto the cultivated tissue and/or onto the body of the patient, the depot being put in place before, during or after the growth process.

The method and the device are suitable in particular for cultivation or production of bones which have a structure similar to the natural structure and have a functional mechanical construction. Such a bone is also referred to below as a genetic living bone.

This genetic living bone is recognized, accepted and integrated as endogenous bone and at the same time spontaneously takes over biomechanical duties. The integration of the implant is made possible by minimization of the cellular physical activities, the phase of endogenous remodeling being spontaneously initiated. The genetic living bone is also used for example for ex vivo cultivation of bone marrow.

The invention is described below on the basis of preferred illustrative embodiments, in particular with reference to the cutivation of bone and the synthesis of structured substances. From these illustrative embodiments, numerous further details and advantages of the invention will be apparent to the skilled person.

### DETAILED DESCRIPTION OF THE INVENTION

Biology is the general science of living things, including anthropology, zoology, botany and microbiology. Within the meaning of the invention, the term biological tissue consequently includes human, animal, plant and microbiological tissue and in particular living tissue. The term biological cells also includes human, animal and plant cells and microrganisms and in particular all living cells.

For the production or cultivation, according to the invention, of biological tissue, in particular of a genetic living bone, a support structure is placed in a specially designed growth chamber and, inside the latter, or before insertion into it, is doted with bone cells. In this bone growth chamber or compartment, nutrient media necessary for bone growth are then made available via a supply system. With suitable temperature control, and by means of transmission of biologically active stimuli or biomechanical impulses, continuously or discontinuously, the biomechanical information stimulating the buildup of bone is transmitted via the support structure. The doted bone cells are biostimulated by this means and can thus perform differentiation. As a result, it is possible for the bone cells to generate differentiated bone and to build this up into biomechanically functional structures. Such a bone constitutes a functionally very valuable bone which can spontaneously take over all biomechanical and cell-biological duties at the implantation site.

For the growth framework according to the invention, various systems are used. In one embodiment, this framework is a nonresorbable auxiliary framework which later remains in the implant and which merely represents a kind of guide route for the genetic living bone. This preferably consists of biocompatible metal, plastic, ceramic or other biocompatible substances. However, this framework can be made of resorbable material. In this case, it is also possible to use plastics, glass or other biocompatible materials. The framework preferably serves only to ensure that the growing genetic living bone has a possibility of settling on the framework, without the latter itself having to bridge any distance. This type of framework then subsequently provides a simple bone with functional bone tissue without special biomechanical properties. Only the resorbable form of the support framework is broken down in the subsequent bone remodeling, so that bone with a genuinely trabecular configuration is able to form after fairly long periods of incorporation.

This is not the case in the production of support frameworks, according to a further preferred embodiment of the invention, which follow biomechanical laws. This kind of framework which can be made from the above materials provides a biomechanically valuable bone already in the genetic living bone growth compartment. In this case, the framework is constructed so that it already has the later inner structure of the desired implant. This is preferably the trabecular structure of living bone, or the cortical structure, or a combination of both structures. Integration of biomechanically supportive structures is likewise possible.

The support frameworks can alternatively also be designed in such a way that they are present during the buildup of the genetic living bone implant but are already eliminated during or after the buildup in the growth compartment, so that the finished implant consists only of genetic living bone. This has in particular the advantage that no foreign materials have to be implanted, i.e. a bone is obtained which is free of foreign material. In this case, the materials for the support framework are preferably materials that promote cell growth, for example cellulose, starch, alcohol compounds, gels or gel-like materials, but also degradable mineral or crystalline inorganic materials, for example calcium phosphate. If the growth framework or support framework consists of such a material which is eliminated during the growth phase, a possibly predeterminable ion exchange with the resulting tissue, for example calcium and sulfate or calcium and phosphate, is suitable for supporting the mineralization of the genetic living bone. This mineralization synergistically completes the development of a biomechanically valuable replacement bone having all the properties of a bone which has developed in vivo.

It is particularly preferable that the growing bone in the growth chamber then also takes the place of the support framework, so that the mechanically valuable structures of the loadable bone can be much more pronounced than if the support framework remains in the implant.

The behavior of the growth compartment during the buildup of the genetic living bone is of particular importance as regards the cultivation of this bone. In natural bone remodeling, a bone can only grow if the biomechanical requirement is forwarded to the defect site. The undifferentiated cells responsible for bone remodeling obey the principle that unrequired bone is broken down, required bone is built up, and old bone replaced. Following this principle, the undifferentiated cells differentiate into bone-forming cells (osteoblasts) and bone-absorbing cells (osteoclasts). For buildup of bone, nutrient media are supplied, and for breakdown of bone, products of degradation are carried away.

In order to stimulate the growth of the genetic living bone during the dwell time in the growth compartment, natural or quasi natural biomechanical stimuli are simulated. These stimuli are effected, for example, by mechanical loading, i.e. a suitable means is used to apply a mechanical tensile, compressive, shearing and/or torsional load, or a combination of these, to the growth framework. The degree of this loading is adapted to the normal mechanical movements of the bone framework in the living body and is therefore correspondingly low, so that, for example, the following methods of transmission are used.

In a first embodiment, the biomechanical stimulus is created and transmitted by the connection of the growth framework in the bone growth compartment by unilateral or bilateral attachment of piezoelectric impulse transmitters. The frequency of the current impulses on the piezoelectric component determines the frequency of the resulting mechanical expansion of the piezo component. The impulse strength here determines the degree of the expansion and thus the intensity of the mechanical load exerted on the growth framework. The pattern of the mechanical impulse can also be suitably controlled. A mechanical stimulus is sent through the growth framework, at each point thereof, which is intended to move the bone cells to the preferred differentiation of the osteoblasts.

In another embodiment, the surface of the growth compartment is subjected to pressure. This pressure is pulsating, intermittent and/or waveshaped. This method of force introduction is slightly slower, but is easier to realize. The variety of structuring obtained by the piezoelectric action is greater however.

In addition, a synergistic effect is obtained by combination of the two aforementioned embodiments, with pressure acting on a piezoelectric layer. The pressure effects the mechanical loading, and, thus initiated, the piezo crystals deliver an electrical impulse which in turn is associated with a contraction or elongation of the crystals. In this connection, use is made of the effect that electrical current impulses can positively influence the biological metabolism. In these cases, the piezo crystals are preferably integrated into the matrix of the growth supports so that an inner mechanical impulse is generated, through the entire implant, in addition to the mechanical impulses delivered from outside.

As an alternative to this, the support framework consists of electrically conductive material. In this way, the stimulation of the cells by electrical currents, fields or voltages is improved.

In a further embodiment, the entire growth compartment is kept in motion by accelerating it and slowing it down. By means of the accelerating and braking forces, an overall force is exerted on the growth framework which likewise represents a biologically effective stimulus or a biomechanical load. However, in this case, not only is the growth support accelerated, but also the cells and the nutrient media. This could disturb the directions of growth. However, this can be influenced positively by such loading with nutrient media. Alternatively or in addition, the biologically effective or biomechanical stimulus is produced using pressure and partial vacuum transmitters. This is particularly cost-effective.

In a further embodiment of the invention, a means for exerting a mechanical force, for example a tension, compression, shear and/or torsion module, is integrated into the support framework. This is especially advantageous for parts of the genetic living bone which are extremely exposed to stresses.

The forming bone tissue is preferably supplied with a suitable nutrient solution. In this connection, the composition is preferably changed, in particular controlled or regulated, as a result of which the bone matrix is offered a selective choice of elements which the bone cells need for bone formation. The growth of the bone cells can also be positively influenced by substances that promote bone growth, for example bone morphogenetic proteins, fibrogens or the like. The use of genetically modified additives or additives produced by genetic engineering is of particular interest in this connection. Ethical aspects can also be taken into consideration here of course.

Depending on the desired tissue type and form, it is possible, when carrying out the method, to balance the intensity and/or nature of the stimuli in a predetermined manner so that the cell degeneration is less than the cell generation. Parameters for influencing this are, for example, temperature, load frequency, load strength and load form.

In the conversion phase, there are in particular two possibilities:

On the one hand, generation of standardized bone from generally compatible cells can be carried out in factory production, particularly for those applications which have to be carried out unplanned. Production tailored to the patient can also be carried out in a factory if a sufficient lead time is available. This is generally done in large chambers, but in the patient-specific case in individual chambers. The development costs for general bone are lower than those for patient-specific bone on account of the batch sizes. Hospitals can be supplied from a central point, and, in cases involving long transport distances, the tissue should be cooled or nutrient media supplied during the transportation.

A second possibility is the production of genetic living bone directly in the hospital, for example in its blood bank or in its cell laboratory. Production can be easily handled using standardized growth chambers and suitable supplies of cells.

A further embodiment of the genetic living bone implants comprises pharmaceutical substances, for example in a depot within the bone. The release of active substances is of very particular importance in medicine. By this means, a pharmaceutical substance generally performs the function of ensuring a protective measure either for the implant or for the surrounding tissue. Infections caused by the conditions prevailing in the operating environment are extremely unlikely in today's hygienic conditions, but they still cannot be ignored. The aim of active substance release is, for example, to prevent inflammations, or to treat diseases such as cancer or tumors, although other functions are also possible. In these circumstances, the duration of release, from short term to long term, and the amount released can be predetermined.

There are likewise various possibilities of charging the genetic living bone with active substance.

In a first method, active substances are introduced into the structured support matrix even before cultivation of the bone cells, by means of this structure being impregnated with the active substance, comprising the active substance or being made up completely or partially of the latter. In this arrangement, the support matrix already releases its active substance to the bone cells and to the nutrient liquid during the cultivation phase. This however leads to a high rate of penetration into the growth tissue.

On the other hand, active substances are particularly preferably added via the nutrient liquid during the growth phase or shortly before or shortly after the growth phase. In some cases it is also feasible to deliver the active substances just shortly before the implantation of the genetic living bone. The amount, concentration and timing of the release can be adapted to the circumstances. In addition to a possible standard charging with active substances, it is also possble for the individual composition to be adapted to patient-specific requirements. The spectrum of the substances in question here lies preferably in the area of antibiotics and cytostatics. However, genetically active substances such as FGF or BMP and others can also be used individually or in combination with other active substances known to the skilled person. In special cases these active substances can also represent so-called trace elements in order, if appropriate, to correct any deficiencies or metabolic disturbances in the body, these in particular being substances implicated in the electrochemical processes, such as electrolytes. Anticoagulants or coagulation promoters such as DTP can also be used, however, in cases of disorders of the blood system. In this type of active substance application, it is advantageous to restrict the area of action to the implant site.

Consequently, with the production methods described above, it is also possible to generate cell-differentiated bone. For this purpose, in a further embodiment, bone cell growth is manipulated by changing the timing of the biomechanical stimuli acting on the growing implant and/or by changing the composition of the nutrient solution. In this way, a bone structure of altered strength and composition is obtained, or other bone substances are added by partially or completely charging the surface of the already grown bone. This completely new generation of implant or active substance is regarded as an embodiment of the genetic living bone having a particularly wide-ranging scope of application.

By following the American model of population coverage in genetic data banks, it is possible, with this invention, to create a worldwide available stock of patient-specific replacement bone.

In the following part of the description, we set out by way of illustration three application examples for cultivation of specific bones and bone constituents starting from clinical requirement profiles.

### EXAMPLE 1

For production of an implant in the form of the described genetic living bone, the model of a femoral neck piece is required, i.e. a connection of cortical and spongy bone framework.

For cultivation, a structural framework of this femoral neck piece is built up from a mass of calcium-enriched collagen using the method of screen printing technology. After it has been produced, this framework is introduced into the growth chamber and the contact with the means for transmitting the biomechanical stimulus is established by placing magnetic pressure plates onto this framework. In this example, the force is introduced by a magnetic field which, through its oscillation form, is idealy adapted for loading of a natural bone. After the system for the growth process has now been made ready, it is inoculated with the growth cells. These cells are at first undifferentiated cells from bone material which differentiate into osteoclasts and osteoblasts during the method. Doting is carried out using a cell solution, by immersing the support matrix into this solution. The undifferentiated cells penetrate into the matrix and settle on the surface. After this, the growth framework or the matrix to be grown over is closed with a cell membrane so that the doted cells cannot migrate away.

The growth chamber is then flushed with a nutrient medium and set in circulation. A timer system ensures regular refilling with fresh nutrient medium and suctioning-off of used nutrient liquid. Ionized calcium and phosphate ions in particular are added to this nutrient liquid since these are required for the mineralization of inorganic bone crystals. In the temperature-controlled growth phase, a dynamic alternating load is applied to the magnetic pressure plates by an externally applied magnetic alternating field. The rising and falling amplitude is in this case adapted to the biological pressure development of a natural movement loading pattern. In the course of cell division, the donor cells multiply in the loaded growth chamber and differentiate, by means of the biomechanical loading, predominantly to osteoblasts. The collagenous support structure is degraded by biochemical solution and integrated in the form of collagenous structures into the growing bone. This integration in turn effects the connection and positioning of the inorganic bone crystal substance. To increase the biomechanical strength of the genetic living bone, the magnetic alternating load has its load amplitude increased at intervals corresponding to the growth rate. At the end of the extracorporeal growth process, the nutrient solution has a pharmacologically active substance added to it, for example an antibiotic, which gives the implant an antibacterial protection. The growth transmission is stopped and the pressure transmission plates are removed from the genetic living bone.

The implant is taken from the growth compartment and stored on an intermediate basis in a transport container at reduced temperatures. The low storage temperature reduces cell death until implantation, so that a genetic living bone with maximum vitality can be implanted. At the time of the operation, the genetic living bone is mechanically adapted to the defect site and then implanted. For improved and more rapid integration, or connection, the implant can be inoculated with fresh substances from the patient, for example blood, bone marrow or the like.

The growth chamber is cleaned and sterilized and is thus made ready for its next use.

### EXAMPLE 2

A genetic living bone produced according to example 1 is intended to supplement a part of the vertebra for optimum integration in bridging a defect in the cervical spine.

For this purpose, the genetic living bone grown is removed from the growth chamber and is coated on its circumferential outer face with a gel of collagen and periosteum cells. A protective membrane of foil is laid over this. This combination is in turn placed in another growth chamber or compartment, supplied from above or below with nutrient solutions and embedded in a muscle-like fleece. A lower torsion plate and an upper torsion plate are then attached to the end faces of the genetic living bone. The torsion plates are set in a slight torsion oscillaton by means of an eccentric drive in order to simulate the turning of the bone in relation to the surrounding muscle tissue. Excited by this simulation, the periosteum cells come together to form a layer which ideally represents a periosteum. The genetic living bone enclosed by periosteum is removed from its envelope and freed from the protective foil.

By means of the periosteal layer, the ideal simulation of the new bone segment can now take over its function in the vertebra.

### EXAMPLE 3

A framework having the outer geometry of a lumbar vertebra is produced from a mixture of poly-D,L-lactide and a crystalline pentacalcium hydroxy(tris)phosphate which is transformed by additives such as titanium oxide to a piezo material. This framework is prepared in the growth chamber in the manner described in examples 1 and 2. However, the introduction of the biomechanical stimulus differs from these examples.

In this example, example 3, one contact plate is placed over the framework and another contact plate below the framework. After doting and nutrient medium supply, an alternating voltage in the frequency range of the resonance frequency of the piezoelectric pentacalcium hydroxy(tris)phosphate crystals is applied to the contact plates. The impulses are introduced through the lactide substance and via the nutrient medium. The piezoelectric contraction and elongation results in a micromechanical loading in all framework parts, which stimulates the bone cells to growth activities. In this process, the lactide is degraded so that after the growth process has been completed the living bone substance remains in the form of the original support matrix. A special feature here is that the piezoelectric pentacalcium hydroxy(tris)phosphate crystals remain in the genetic living bone and, after implantation, conversely deliver an additional impulse to the organism, now in vivo. As a result of the biomechanical loading of the bone by movement patterns, these crystals deliver a small current impulse which is supplied to the surrounding tissue. This current impulse in turn acts positively on the bone growth and bone regeneration (similar to what is called electrotherapy). In this way, an additional aid to integration of the implant into the body is assured.

Alternative embodiments of the invention concern the production or generation of other functional tissue including organ sections, organ constituents, whole organs, for example internal organs, body parts and/or generally functional and/or structured cell conglomerates, for example cartilage, blood vessels, ears, noses, skin, etc. The cultivation of structured tissue is also an important advance for production of other functional tissue types.

Examples of these are the cultivation of cartilaginous tissue, such as the nasal septum, or the anvil, hammer and stirrup of the auditory canal, or intervertebral disks of the spinal column.

Further examples of functional components which can be cultivated according to the invention are vessel walls, whole vessel sections, the walls of the fallopians, ureter and urethra, or the intestinal walls.

By means of a selective tissue modification, such components can be combined in onlay techniques with other tissue types, so that functional connection to other organ areas or tissue areas, such as muscle groups or even nerves, is possible.

In a particularly preferred development of the invention, even multifunctional component groups can be produced as a body replacement part. In this case, the biomechanical stimulus preferred for bone tissue is replaced or supplemented by other biological initiators.

In a further embodiment, combined effects are used in the growth compartments with different aims. For example, vital bone marrow is cultivated from donor cells. These cells can derive from a fresh specimen, for example from the patient himself or from a compatible donor. Moroever, it is possible to generate bone marrow from autologous cells obtained from babies or infants and stored in the frozen state, in the same way as in gene banks or sperm banks.

In this combined method, the simulated growth localization is imparted to the bone marrow cells via a precultivated bone, in some cases in the biomechanical arrangement of the simulated spinal column or simulated marrow bone. Environments produced according to the invention then permit the cultivation of bone marrow in vitro.

This opens up new possibilities in the prevention of bone marrow diseases such as leukemia, cancer or tumors. The time aspect of the generation is of importance here, because first the environment is cultivated and therafter the marrow. One of the greatest problems of the already known methods is the limited availability of donor marrow. Therefore, a particular advantage of the invention is that such cultivation can be carried out specifically for the patient and in almost unlimited amounts.

Such donor cell cultivation is cost effective in terms of the labor involved and the amount of material involved and also in view of the costs of storing the donor cells, optimally for life.

The availability aspect in the event of a sudden outbreak of such a disease is also seen as a positive contribution to medical prevention and treatment.

It will be evident to the skilled person that the invention is not limited to the embodiments described above, and that it can instead be varied in a number of ways without departing from the spirit of the invention.

1. A method for producing biological tissue in a growth chamber, in particular for transplantation into or onto a human or animal body, comprising
applying biological cells to a growth framework, said growth framework defining an initial form of the tissue to be produced,
arranging the biological cells and the growth framework in the growth chamber, and
exerting a biologically active stimulus on the growth framework and/or on the biological cells.

2. The method as claimed in claim 1, wherein the stimulus corresponds to or is at least similar to a stimulus to which the tissue is naturally exposed in or on the body.

3. The method as claimed in claim 1 or 2, wherein structured and/or functional biological tissue is produced.

4. The method as claimed in one of the preceding claims, wherein growth, form, function, structure and/or nature of the biological tissue is influenced by the stimulus or a sequence of stimuli.

5. The method as claimed in one of the preceding claims, wherein different stimuli and/or different kinds of stimuli are exerted and cell-differentiated tissue sections are produced.

6. The method as claimed in one of the preceding claims, wherein the growth framework, at the start of the method, essentially defines only an initial form of the tissue to be produced.

7. The method as claimed in one of the preceding claims, wherein an organ, a bone, a cartilage, a blood vessel, periosteum, or a functional combination of these, is produced.

8. The method as claimed in one of the preceding claims, wherein the grown tissue essentially determines the biomechanical properties of the arrangement of tissue and growth framework.

9. The method as claimed in one of the preceding claims, wherein a framework is used comprising resorbable material, in particular biological material or cells.

10. The method as claimed in one of the preceding claims, wherein a framework is used comprising a material that promotes cell growth, in particular cellulose, starch, alcohol compounds, gel and/or gel-like material.

11. The method as claimed in one of the preceding claims, wherein the growing tissue takes the place of the growth framework.

12. The method as claimed in one of the preceding claims, wherein a framework is used comprising nonresorbable material and/or electrically conductive material.

13. The method as claimed in one of the preceding claims, wherein the framework is separated from the tissue which has grown from the biological cells on the framework.

14. The method as claimed in one of the preceding claims, wherein a pharmacologically active substance, preferably a growth-promoting substance, particularly preferably a bone morphogenetic protein, a fibrogen and/or a genetically modified substance is added.

15. The method as claimed in one of the preceding claims, wherein a depot of a pharmacologically active substance is placed on and/or in the growth framework and/or the tissue.

16. The method as claimed in claim 15, wherein the pharmacologically active substance is released after transplantation of the tissue into or onto the body.

17. The method as claimed in one of the preceding claims, wherein mechanical, electrical, magnetic, chemical, olfactory, acoustic and/or optical stimuli are exerted on the growth framework and/or on the tissue.

18. The method as claimed in one of the preceding claims, wherein stimuli which can be changed in terms of their timing, in particular discontinuous stimulating impulses and/or periodic stimuli, are exerted on the growth framework and/or on the biological cells.

19. The method as claimed in one of the preceding claims, wherein biologically active stimuli are exerted with a piezoelectric material.

20. The method as claimed in one of the preceding claims, wherein a piezoelectric means is arranged on and/or in the growth framework.

21. A growth framework for producing biological tissue, in particular for transplantation into or onto a human or animal body, and in particular for use of the method as claimed in one of the preceding claims, in which
the growth framework defines an initial form of the tissue to be produced,
the growth framework can be arranged in a growth chamber,
biological cells can be applied to the growth framework, and
a biologically active stimulus can be exerted on the growth framework and/or on the biological cells.

22. The growth framework as claimed in claim 21, wherein biological tissue with functional structure can be produced or cultivated.

23. The growth framework as claimed in one of the preceding claims, wherein the growth framework essentially defines only an initial form of the biological tissue.

24. The growth framework as claimed in one of the preceding claims, wherein different stimuli and/or different kinds of stimuli can be exerted.

25. The growth framework as claimed in one of the preceding claims, wherein growth, form, function, structure and/or nature of the tissue can be influenced by the stimulus or a sequence of stimuli.

26. The growth framework as claimed in one of the preceding claims, wherein the biological tissue comprises an organ, a bone, a cartilage, a blood vessel, periosteum, or a functional combination of these.

27. The growth framework as claimed in one of the preceding claims, wherein the grown tissue essentially determines the biomechanical properties of the arrangement of tissue and growth framework.

28. The growth framework as claimed in one of the preceding claims, wherein resorbable material is used, in particular biological material or cells.

29. The growth framework as claimed in one of the preceding claims, wherein a material that promotes cell growth is used, in particular cellulose, starch, alcohol compounds, gel and/or gel-like material.

30. The growth framework as claimed in one of the preceding claims, wherein its place can be taken by the growing tissue.

31. The growth framework as claimed in one of the preceding claims, wherein it comprises nonresorbable material and/or electrically conductive material.

32. The growth framework as claimed in one of the preceding claims, wherein it can be separated from the tissue which has grown from the biological cells on the framework.

33. The growth framework as claimed in one of the preceding claims, wherein a depot of a pharmacologically active substance can be placed on and/or in the growth framework and/or the tissue.

34. The growth framework as claimed in claim 33, wherein, after transplantation of the tissue into or onto a body, the pharmacologically active substance can be released thereto.

35. The growth framework as claimed in one of the preceding claims, wherein the biologically active stimulus comprises mechanical, electrical, magnetic, chemical, olfactory, acoustic and/or optical stimuli.

36. The growth framework as claimed in one of the preceding claims, wherein the biologically active stimulus comprises stimuli which can be changed in terms of their timing, in particular discontinuous stimulating impulses and/or periodic stimuli.

37. The growth framework as claimed in one of the preceding claims, comprising a means for stimulus generation, in particular a piezoelectric material.

38. A device for producing biological tissue, in particular for transplantation into or onto a human or animal body, and in particular for use of the method as claimed in one of the preceding method claims, said device comprising
a growth chamber,
a growth framework, in particular as claimed in one of claims 21 through 37, arranged in the growth chamber, biological cells arranged on the growth framework, and
a means for generating biologically active stimuli and for exerting the stimuli on the growth framework and/or the biological cells.

39. The device as claimed in claim 38, wherein the stimuli correspond to or are similar to stimuli to which the tissue is naturally exposed in or on the body.

40. The device as claimed in claim 38 or 39, wherein a pharmacologically active substance, preferably a growth-promoting substance, particularly preferably a bone morphogenetic protein, a fibrogen and/or a genetically modified substance can be added.

41. The device as claimed in one of the preceding claims, having a means for generating mechanical, electrical, magnetic, chemical, olfactory, acoustic and/or optical stimuli.

42. The device as claimed in one of the preceding claims, having a means for generating stimuli which can be changed in terms of their timing, in particular stimulating impulses and/or periodic stimuli.

43. The device as claimed in one of the preceding claims, having a means for generating biologically active stimuli, in particular mechanical and/or electrical stimuli, using the piezoelectric effect.

44. A biological tissue which can be produced or is produced by the method as claimed in one of claims 1 through 20, can be produced or is produced with the growth framework as claimed in one of claims 21 through 37, and/or can be produced or is produced in the device as claimed in one of claims 38 through 43, said tissue being cultivated and having a predetermined form, structure and/or functionality.

45. An implant or onplant for implanting or onplanting in or on a human or animal body, comprising the tissue as claimed in claim 44.

46. The implant or onplant as claimed in claim 45, in which said tissue further comprises an active substance depot for releasing a pharmacologically active substance into or onto the body.

47. A bone which can be produced or is produced by the method as claimed in one of claims 1 through 20, can be produced or is produced with the growth framework as claimed in one of claims 21 through 37, and/or can be produced or is produced in the device as claimed in one of claims 38 through 43, said bone having a trabecular and/or cortical structure.

48. The use of the method as claimed in one of claims 1 through 20, of the growth framework as claimed in one of claims 21 through 37, or of the device as claimed in one of claims 38 through 43, for producing or cultivating bone marrow in a bone outside or inside a living body.
